(19) United States
(12) Patent Application Publication
(10) Pub. No.: US 2025/0127839 A1
(43) Pub. Date: Apr. 24, 2025

(54) METHOD TO INCREASE THE CONCENTRATION OF SHATAVARIN I AND SHATAVARIN IV IN EXTRACT FROM ROOTS

(71) Applicant: WALERIA HEALTHTECH PVT LTD, Hyderabad (IN)

(72) Inventor: Nameet Baldwa, Telangana (IN)

(73) Assignee: WALERIA HEALTHTECH PVT LTD, Hyderabad (IN)

(21) Appl. No.: 18/921,166

(22) Filed: Oct. 21, 2024

(30) Foreign Application Priority Data
Oct. 20, 2023 (IN) 202341071648

(51) Int. Cl. A61K 36/8965; A61K 31/7032; A61K 31/7048; A61P 25/22; A61P 43/00

(52) U.S. Cl. CPC A61K 36/8965; A61K 31/7032; A61K 31/7048; A61P 25/22; A61P 43/00; A61K 2236/333; A61K 2236/37; A61K 2236/39; A61K 2236/51

(57) ABSTRACT

The present invention provides a method to increase the concentration of shatavarin I and shatavarin IV in an extract derived from roots of A. racemosus. The method comprises the steps of adding roots of A. racemosus in a solvent, wherein the solvent is methanol: water mixture or ethanol: water mixture; stirring the roots in the solvent at 90° C.; filtering to separate the residue from the filterate; evaporating the residue to dry it to obtain dried extract comprising of shatavarin I and shatavarin IV at a concentration of 4-6.5%.

### PRIORITY PARAGRAPH

The Application claims priority to Indian Application No. 202341071648 filed on 20th October 2023 titled “Method to increase the concentration of shatavarin i and shatavarin iv in extract from roots”. The priority application is fully incorporated herein by reference.

### FIELD OF THE INVENTION

The present invention provides a method or process to increase the yield or of Shatavarins I and IV in extracts derived from roots of Asparagus racemosus willd (shatavari).

### BACKGROUND OF THE INVENTION

Asparagus sp. belongs to the family of Liliaceae. These are medicinal shrubs valued for their medicinal properties. More than 300 sp. of Asparagus are known

Asparagus racemosus willd is well known sp. from which many medicinal constituents are extracted. The roots of A. racemosus are bitter in taste and mostly indigestible. In the Unani medicinal system, it is used as laxative and as other agents. The Shatavari plant has been used in ayurveda for various conditions including its use as a galactagogue-to increase milk secretion during lactation. It is also used as a general tonic, and as an aphrodisiac. Root extracts of A. racemosus are also known for antioxidant, antibacterial, antiulcer, and anticancer properties. There are many chemical constituents derived from different plant products of A. racemosus and among the chemical constituents derived from roots are class of compounds called steroidal saponins, more specifically shatavarins. Shatavarins I-X are known of which shatavarin IV is most reported and studied.

Mostly the dried roots of shatavari are used directly for their medicinal properties and these compositions rely on the crude extracts instead of use of active agents with higher concentration and purity levels.

702/MUM/2005 discloses a herbal medicinal composition with therapeutically effective amounts of dried roots of Chlorophytum species, Withania species, Asparagus species, and Gokhru (Tribulus Terrestris). Said invention uses dried roots of various herbal plants and does not extract the secondary metabolites which act the active agents for maximum efficacy.

However, several attempts have been made to extract the active agents to increase the efficacy of the final product in the medicinal compositions

Some of the prior art which provide extraction methods are provided below:

IN201821031061 provides a method for the maximum production of tuberous roots of Shatavari using bioelicitation technique. The invention describes a method to cultivate the roots and to extract the secondary metabolite Shatavarin IV with increased yield and concentration.

Gohel et al. in their publication titled “Isolation and characterization of Shatavarin IV from root of Asparagus racemosus willd” describe a method to isolate Shatavarin IV from crude root powder of Asparagus racemosus willd. Said method required the use of several solvents which are expensive, harmful and corrosive in nature such as Hexane, and 90% Methanol for maceration of the roots. Then the methanolic extract was dried and redissolved in 10% methanol followed by successive portioning with Chloroform, Ethyl acetate and n-Butanol. The n-Butanol extract was further dried and dissolved in 90% methanol to be used for column chromatographic isolation of Shatavarin IV. Said method extracted 401.1 mg Shatavarin IV from 250 g of crude root powder of Asparagus racemosus willd. This amounts to only 0.16% of the compound with only 66% purity level. Said method not only uses several solvents for extraction but said method does not provide enough concentration of Shatavarin IV with good purity. This again reduces the efficacy of the final product in a medicinal composition.

On the other hand, Mitra et. al. in their publication titled “Shatavarins (containing Shatavarin IV) with anticancer activity from the roots of Asparagus racemosus” have reported to have isolated 84.69% pure shataravin IV extract containing 5.05% shatavarin IV which showed potent cytotoxicity against cancerous mammalian secondary cell lines. However, the method involved use of several solvents. Most of the literature available only provides methods to isolate shatavarin IV and there is no known publication which provides an efficient method to extract shatavarin I with high purity and in high concentrations. Further, though pure forms of shatavarin IV are provided, so far there is no pure form of shatavarin I in the market to assess the efficiency and efficacy of shatavarin I present in herbal and/or medicinal compositions.

Taking the drawbacks of the prior art, the present invention provides a method to isolation shatavarin I and IV with high purity levels and high concentration from A. racemosus.

### OBJECTS OF THE INVENTION

The main object of the invention is to provide a method to increase the concentration of shatavarin I and shatavarin IV in an extract derived from roots of A. racemosus.

Anther object of the invention is to provide a composition of extract comprising of shatavarin I and shatavarin IV at a concentration of 4-6.5%.

Yet another object of the invention is to provide a composition of extract comprising of shatavarin I and shatavarin IV which has application in managing menopausal symptoms in women.

### SUMMARY OF THE INVENTION

The present invention provides a method to increase the concentration of shatavarin I and shatavarin IV in an extract derived from roots of A. racemosus. Said method enables extraction of shatavarin I and shatavarin IV at a concentration of 4-6.5% in total from roots of A. racemosus. Said method comprises the steps of adding roots of A. racemosus in a solvent; stirring the roots in the solvent at 90° C.; filtering to separate the residue from the filtrate; repeating the above steps with the obtained residue at least 3 more times to obtain final residue; evaporating the final residue to dry it at a temperature of boiling point of the solvent; and dried extract comprising of shatavarin I and shatavarin IV at a concentration of 4-6.5%. Said method utilizes solvent selected from the group consisting of mixture of methanol and water, and mixture of ethanol and water. Said mixture of methanol and water or ethanol and water are at a ratio selected from the group consisting of 80:20, 70:30, 60:40, 50:50, 40:60, more specifically, a ratio of 70:30.

The present invention further provides a method to extract shatavarin I and shatavarin IV at a concentration of 5.86% from roots of A. racemosus, wherein, the method comprises the steps of adding roots of A. racemosus in solvent ethanol and water in a ratio of 70:30; stirring the roots in the solvent at 90° C.; filtering to separate the residue from the filtrate; repeating the above steps with the obtained residue at least 3 more times to obtain final residue; evaporating the final residue to dry it at 65° C. (boiling point of the solvent); and dried extract comprising of shatavarin I and shatavarin IV. Said dried extract comprised of 4.92% of shatavarin I and 0.94% of shatavarin IV.

The present invention also provides a method to extract shatavarin I and shatavarin IV at a concentration of 6.02% from roots of A. racemosus, wherein, the method comprises the steps of adding roots of A. racemosus in solvent methanol and water in a ratio of 70:30; stirring the roots in the solvent at 90° C.; filtering to separate the residue from the filtrate; repeating the above steps with the obtained residue at least 3 more times to obtain final residue; evaporating the final residue to dry at 65° C. (boiling point of the solvent); and dried extract comprising of shatavarin I and shatavarin IV. Said dried extract comprised of 4.75% of shatavarin I and 1.27% of shatavarin IV.

The use of ethanol and water mixture as a solvent at 70:30 ratio provides slightly lesser amount of total shatavarin I and shatavarin IV, 5.86%, in the dried extract compared to methanol and water solvent at 70:30 ratio, 6.02% of total shatavarin I and shatavarin IV. Nevertheless, ethanol and water mixture as a solvent at 70:30 ratio is more favourable as methanol is suspected to have carcinogenic and other toxic properties.

### BRIEF DESCRIPTION OF THE DRAWINGS

FIG. 1A provides the chemical structure of shatavarin I compound (molecular formula C₅₁H₈₆O₂₃).

FIG. 1B provides the chemical structure of shatavarin IV compound (molecular formula C₄₅H₇₄O₁₇).

FIG. 2 provides a graphical representation of concentration of total shatavarin I and shatavarin IV obtained from roots of A. racemosus using various solvents described in Table 1.

FIG. 3 provides a graphical representation of summary of the Utian Quality of Life (UtQoL) scale as measured on day 30 and day 60 after treatment of patients with either placebo or active ingredient (Aspurus)—the shatavari extract derived from roots of A. racemosus having 5.86% total shatavarin I and shatavrin IV.

FIG. 4 provides a graphical representation of DASS-21 score (Depression, Anxiety and Stress Scale 21 Items score) at baseline, day 30, and day 60 visits for the evaluation of depression, anxiety, and stress after treatment of patients with either placebo or active ingredient (Aspurus)—the shatavari extract derived from roots of A. racemosus having 5.86% total shatavarin I and shatavrin IV.

FIG. 5 provides a graphical representation of summary of the five-point Likert scale study results which compares the outcomes recorded for the parameters (hot flashes, night sweats, anxiety, fatigue, insomnia, and mood swings) obtained in patients treated with placebo or active ingredient (Aspurus)—the shatavari extract derived from roots of A. racemosus having 5.86% total shatavarin I and shatavrin IV at the baseline, day 30, and day 60 visits.

### DETAILED DESCRIPTION OF THE INVENTION

In the main embodiment, the invention provides a method to increase the concentration of shatavarin I and shatavarin IV in an extract derived from roots of A. racemosus. Said method comprises the steps of:
a) adding roots of A. racemosus in a solvent;
b) stirring the roots in the solvent at 90° C. for maceration;
c) filtering the macerated mixture to separate the residue from the filtrate;
d) repeating steps a)-c) for at least 3 more times to obtain the final residue
e) evaporating the residue at a boiling point of the solvent to obtain dried extract comprising of shatavarin I and shatavarin IV at a concentration of 4-6.5%.

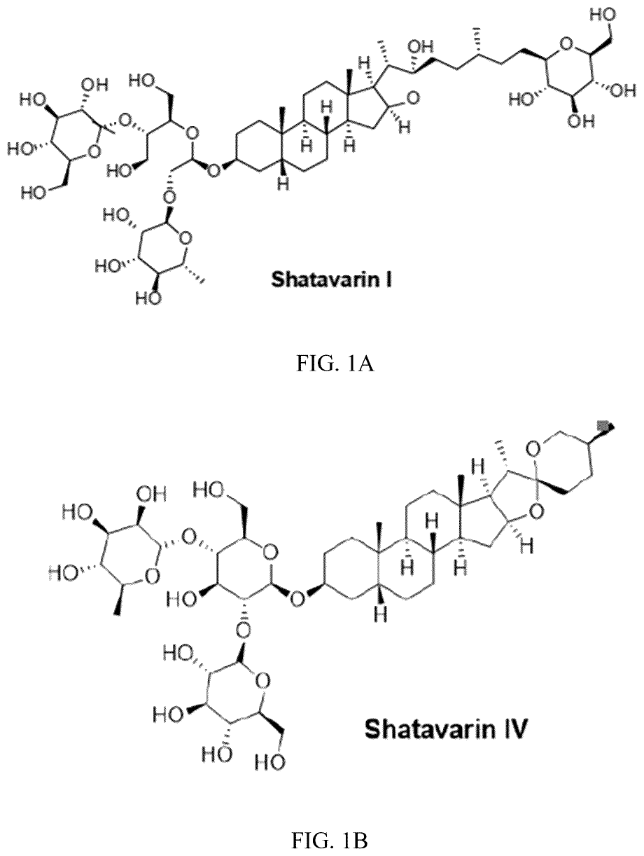

FIG. 1A provides the chemical structure of shatavarin I and FIG. 1B provides the chemical structure of shatavrin IV.

Said method utilizes solvent selected from the group consisting of mixture of methanol and water, and mixture of ethanol and water. Said mixture of methanol and water or ethanol and water are at a ratio selected from the group consisting of 80:20, 70:30, 60:40, 50:50, 40:60, more specifically, a ratio of 70:30.

In another embodiment, the invention provides a composition of extract derived from roots of A. racemosus, wherein, the concentration of shatavarin I and shatavarin is between 4-6.5%. The composition further comprises at least 4% shatavarin I, and at least 0.9% of shatavarin IV.

In yet another embodiment, the invention provides a method to extract shatavarin I and shatavarin IV from roots of A. racemosus comprising the steps of:
a) adding roots of A. racemosus in solvent, ethanol and water mixture at a ratio of 70:30;
b) stirring the roots in the solvent at 90° C. for maceration;
c) filtering the macerated mixture to separate the residue from the filtrate;
d) repeating steps a)-c) for at least 3 more times to obtain the final residue
e) evaporating the residue at 65° C. to obtain dried extract comprising of shatavarin I and shatavarin IV.

Said method enables extraction of total shatavarin 1 and shatavarin 4 at a concentration of 5.86%, comprising of 4.92% of shatavarin I and 0.94% of shatavarin IV in the extract.

In yet another embodiment, the invention provides a method to extract shatavarin I and shatavarin IV from roots of A. racemosus comprising the steps of:
a) adding roots of A. racemosus in solvent, methanol and water mixture at a ratio of 70:30;
b) stirring the roots in the solvent at 90° C. for maceration;
c) filtering the macerated mixture to separate the residue from the filtrate;
d) repeating steps a)-c) for at least 3 more times to obtain the final residue
e) evaporating the residue at 65° C. to obtain dried extract comprising of shatavarin I and shatavarin IV.

Said method enables extraction of total shatavarin 1 and shatavarin 4 at a concentration of 6.02%, comprising of 4.75% of shatavarin I and 1.27% of shatavarin IV in the extract.

Said composition comprising of shatavarin I and shatavarin IV with 4-6.5% yield has application in treating Menopause and its symptoms, Decreased lactation/Galactagogue, Poly Cystic Ovarian Syndrome (PCOS), Male & Female Infertility, Lowered Immunity, Early ageing, Stress, Anxiety, and general wellbeing.

### Examples

### 1. Solvents for Extraction

Table 1 provides the list of solvent which were used for extraction of shatavarin rich extract from the roots of A. racemosus.

TABLE 1

| S. No. | Solvent | Ratio |
|---|---|---|
| 1. | Water | 100% |
| 2. | Water:Acetone | 40:60 |
| 3. | Water:Ethanol | 40:60 |
| 4. | Chloroform:Water:Methanol | 7:0.5:2.5 |
| 5. | Methanol:Chloroform | 70:30 |
| 6. | Methanol:Chloroform | 60:40 |
| 7. | Ethyl Acetate:Water:Methanol | 7.2:0.8:2 |
| 8. | Ethyl Acetate:Water:Methanol | 7:1:2 |
| 9. | Ethyl Acetate:Water:Methanol | 5:1.1:1 |
| 10. | Methanol:Water | 80:20 |
| 11. | Methanol:Water | 70:30 |
| 12. | Methanol:Water | 60:40 |
| 13. | Methanol:Water | 40:60 |
| 14. | Methanol:Water | 50:50 |
| 15. | Ethanol:Water | 80:20 |
| 16. | Ethanol:Water | 70:30 |
| 17. | Ethanol:Water | 60:40 |
| 18. | Ethanol:Water | 40:60 |
| 19. | Ethanol:Water | 50:50 |

### 2. Extraction Process Using Ethanol and Water Solvent

The following process was employed to extract shatavarin rich extract from roots of A. racemosus. The method comprised the steps of:
a) Taking 10 kgs of Shatavari roots in a reactor along with 400 ltrs of ethanol and water mixture solvent;
b) continuously stirring the mixture for 4 hours at 90° C. for maceration;
c) filtering the macerated mixture to separate residue from the filtrate;
d) mixing the semi-liquid residue in the respective solvent and following steps a) to c) at least 3 times;
e) drying the residue in an evaporator for at least 4 hours at 65° C. to obtain semi-liquid residue;
f) final drying of the semi-liquid in a drier at a temperature of 90° C. to obtain the final dried extract which is rich in shatavarins; and
g) grinding the dry extract to form powder.

Table 2 provides the efficiency of different ratios of ethanol and water mixture used as solvent to extract shatavarins from roots of A. racemosus.

TABLE 2

| | | | Shatavarin | Shatavarin | Total % |
|---|---|---|---|---|---|
| S | | Solvent | IV (S4) | I (S1) | (S1 + S4) in |
| No | Solvent | ratio | yield % | yield % | extract |
| 1 | Ethanol:Water | 80:20 | 0.41 | 3.64 | 4.05 |
| 2 | Ethanol:Water | 70:30 | 0.94 | 4.92 | 5.86 |
| 3 | Ethanol:Water | 60:40 | 1.48 | 3.71 | 5.19 |
| 4 | Ethanol:Water | 40:60 | 1.41 | 2.92 | 4.33 |
| 5 | Ethanol:Water | 50:50 | 1.32 | 3.14 | 4.46 |

### 3. Extraction Process Using Methanol and Water Solvent

The following process was employed to extract shatavarin rich extract from roots of A. racemosus. The method comprised the steps of:
h) Taking 10 kgs of Shatavari roots in a reactor along with 400 ltrs of methanol and water mixture solvent;
i) continuously stirring the mixture for 4 hours at 90° C. for maceration;
j) filtering the macerated mixture to separate residue from the filtrate;
k) mixing the semi-liquid residue in the respective solvent and following steps a) to c) at least 3 times;
l) drying the residue in an evaporator for at least 4 hours at 65° C. to obtain semi-liquid residue;
m) final drying of the semi-liquid in a drier at a temperature of 90° C. to obtain the final dried extract which is rich in shatavarins; and
n) grinding the dry extract to form powder.

Table 3 provides the efficiency of different ratios of methanol and water mixture used as solvent to extract shatavarins from roots of A. racemosus.

TABLE 3

| S | | Solvent | % S4 | % S1 | Total % |
|---|---|---|---|---|---|
| No | Solvent | ratio | yield | yield | (S1 + S4) |
| 1 | Methanol:Water | 80:20 | 0.54 | 3.59 | 4.13 |
| 2 | Methanol:Water | 70:30 | 1.27 | 4.75 | 6.02 |
| 3 | Methanol:Water | 60:40 | 1.57 | 3.69 | 5.26 |
| 4 | Methanol:Water | 40:60 | 1.68 | 2.83 | 4.51 |
| 5 | Methanol:Water | 50:50 | 1.67 | 3.02 | 4.69 |

### 4. Extraction Process Using Other Solvent

When water and acetone solvent was used at ratio of 40:60 to extract shatavarins from roots of A. racemosus by the method explained in example 1 following was the shatavarins concentration in the extract:
% S1 yield—0%
S4 yield—0.21
Total % (S1+S4)—0.21

When water was used as solvent to extract shatavarins from roots of A. racemosus by the method explained in example 1 following was the shatavarins concentration in the extract:
% S1 yield—0.49%
S4 yield—0.18
Total % (S1+S4)—0.67

When Methanol: Chloroform was used as solvent to extract shatavarins from roots of A. racemosus by the method explained in example 1 following was the shatavarins concentration in the extract as shown in Table 4:

TABLE 4

| S | | Solvent | % S4 | % S1 | Total % |
|---|---|---|---|---|---|
| No | Solvent | ratio | yield | yield | (S1 + S4) |
| 1 | Methanol:Chloroform | 70:30 | 2.81 | 0.12 | 2.93 |
| 2 | Methanol:Chloroform | 60:40 | 2.1 | 0.12 | 2.22 |

When Ethyl Acetate:Water:Methanol was used as solvent to extract shatavarins from roots of A. racemosus by the method explained in example 1 following was the shatavarins concentration in the extract as shown in Table 5:

TABLE 5

| S | | Solvent | % S4 | % S1 | Total % |
|---|---|---|---|---|---|
| No | Solvent | ratio | yield | yield | (S1 + S4) |
| 1 | Ethyl Acetate:Water:Methanol | 7.2:0.8:2 | 0.91 | 0.72 | 1.63 |
| 2 | Ethyl Acetate:Water:Methanol | 7:1:2 | 0.72 | 0.81 | 1.53 |
| 3 | Ethyl Acetate:Water:Methanol | 5:1.1:1 | 0.64 | 0.92 | 1.56 |

Of all the solvents used the solvent methanol: water mixture in the ratio of 70:30 provided the best results. The boiling point of methanol is 65° C. which was used as the temperature for evaporation of solvent from residue. Said solvent enabled extraction of shatavarin I and shatavarin IV at a concentration of at least 5% from the roots of A. racemosus. 2.4 kgs of final dried extract powder with shatavarin I and shatavarin IV at a concentration of at least 5% was derived from 10 kg of roots of A. racemosus.

When Chloroform:Water:Methanol [ratio—7:0.5:2.5] was used as solvent to extract shatavarins from roots of A. racemosus by the method explained in example 1 following was the shatavarins concentration in the extract:
% S1 yield—0.71%
S4 yield—0.80
Total % (S1+S4)—1.51

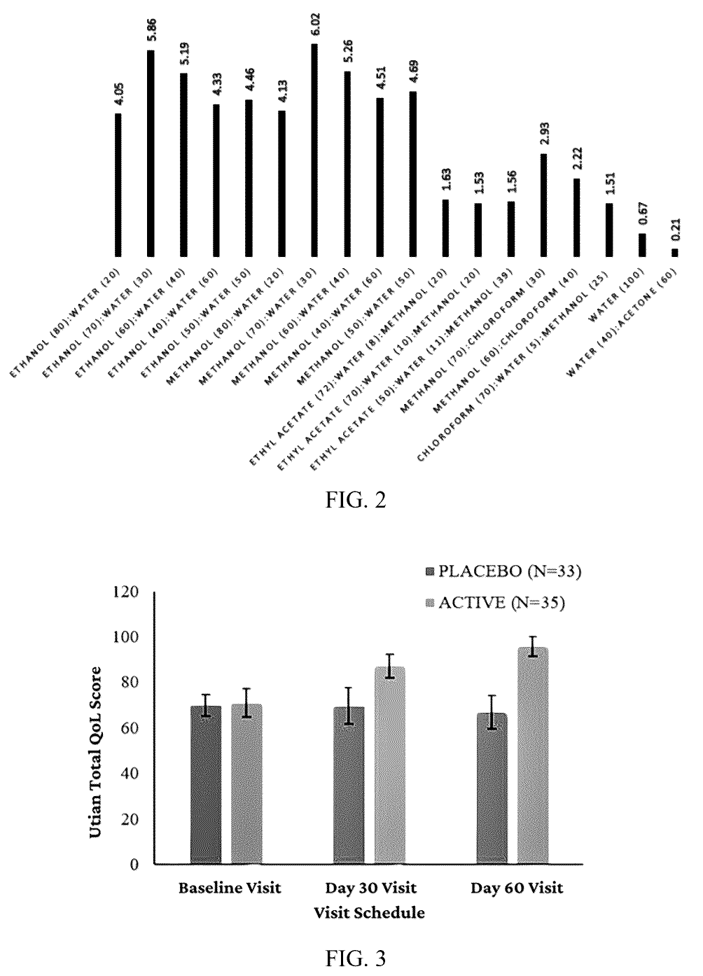

FIG. 2 shows a graphical representation of concentration of total shatavarin I and shatavarin IV obtained from roots of A. racemosus using various solvents by the method described in example 1. Even though extraction with methanol and water mixture (70:30 ratio) yields slightly better results compared to ethanol and water mixture (70:30 ratio), using ethanol is preferable due to suspected carcinogenic properties of methanol. The marginal increase in yield does not justify the potential health risks associated with methanol. Therefore, ethanol seems to be the best solvent of choice for extraction, balancing both efficacy and safety.

### 5. Effect of Shatavarin Extract on Menopausal Symptoms

Menopause is a pivotal and inevitable physiological milestone in the lives of women. The global population of postmenopausal women is on the rise, constituting 26% of all females under the age of 50. Notably, there is a 21% increase in the occurrence of early-age menopause. The sudden or gradual decline in estrogen levels creates an imbalance in the hypothalamic-pituitary-ovarian (HPO) axis. This can lead to failure of endometrial development causing irregular menstrual cycles and progressively contributing to the halt of periods. During menopause, the intricate interplay of changing hormonal levels, specifically estrogen and progesterone, renders women more vulnerable to a spectrum of symptoms. These symptoms encompass hot flashes, vaginal dryness, disturbed sleep, insomnia, urogenital infections, osteoporosis, early onset of coronary heart disease, anxiety, depression, diabetes, and cognitive difficulties. Collectively, these symptoms are comprehensively categorized as menopausal symptoms. Approximately, 75% of women experience perimenopausal or menopausal symptoms due to estrogen deficiency. The transition from a perimenopausal state to menopause is often troublesome, mainly because of menopausal symptoms.

Menopausal symptoms may be mitigated to a certain degree through the use of local or systemic exogenous estrogen administration via hormone replacement therapy (HRT). However, the utilization of HRT is linked with increased risks of complications, such as breast cancer, heart disease, and thromboembolism, as well as other potential side effects. Hence, an alternative therapy devoid of such risks or reduced risks is warranted to manage menopausal symptoms. In a systematic review, it was reported that across all surveys, more than 50% of the women used complementary and alternative medicine to treat their menopausal symptoms specifically. Ayurvedic treatment principles have huge potential for managing menopausal symptoms.

The following clinical study showed the effect of shatavari extract on menopausal symptoms.

### A. Study Design

This was an eight-week, multicenter, interventional, prospective, randomized, double-blind, placebocontrolled, parallel clinical trial structured to compare the safety and efficacy of the test active ingredient with the placebo in the management of menopausal symptoms and regulation of the HPO axis in pre and postmenopausal women. The trials were conducted at two locations in Vijayawada and Guntur, Andhra Pradesh, India. The study was conducted in two hospitals in Andhra Pradesh with sample size n=40 and n=30.

The test product used for the present study was Aspurüs™ which is the shatavari extract derived from above examples (Example 2) using ethanol as solvent which provided an extract having 5.86% total shatavarin I and shatavrin IV. The allergen-free test product was tested for heavy metals such as lead, arsenic, cadmium, and mercury following the Association of Official Agricultural Chemists 2015.01 standard methodology.

### B. Primary Outcome

The primary outcome of the study was to observe the change in the total UQOL score compared to the baseline. FIG. 3 provides a graphical representation of summary of the UQOL scale as measured on day 30 and day 60. An increase in the total score by 16.46 (23.19%) and 24.94 (35.13%) on day 30 and day 60, respectively, was observed in the active (test product) group. Statistically significant intragroup differences were observed on both day 30 (p<0.0001) and day 60 (p<0.0001).

### C. Secondary Outcome

The active (test group) group was found to be better in therapeutic response compared to the placebo (MCC) group in each of the occupational UQOL score, health UQOL score, emotional UQOL score, and sexual UQOL score as shown in Table 6. There was a significant change in the occupational UQOL score, health UQOL score, emotional UQOL score, and sexual UQOL score with a comparative p-value of 0.2063, 0.5678, 0.2751, and 0.7993, respectively, at baseline visit to <0.0001 at the final visit (visit 5) in the active group. An intragroup significant change (p<0.0001) in the mean scores of all the domains of UQOL in the active group was observed.

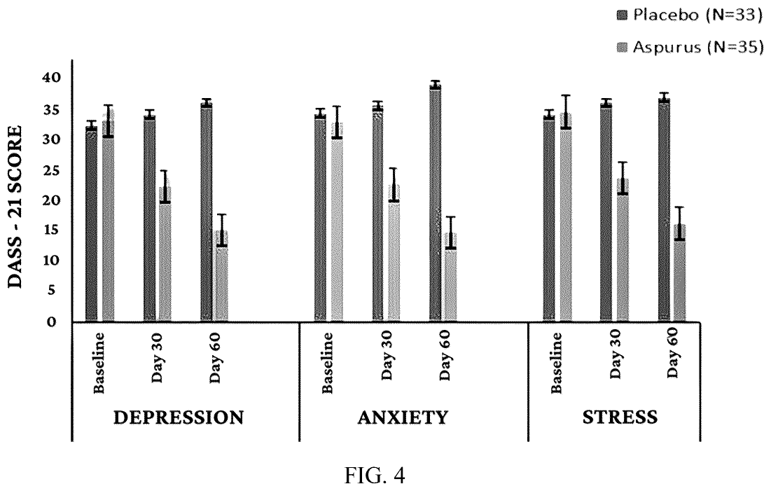

The active group was found to be better in therapeutic responses compared to the placebo (MCC) group as shown in Table 7 and depicted in FIG. 4 in each of the Depression, Anxiety, and Stress scores of DASS-21 with a p-value of 0.5708, 0.0938, and 0.6868, respectively, at the baseline visit to statistically significant outcomes (p<0.0001) at the final visit (visit 5).

TABLE 6
Summary Statistics of Utian Quality of Life Scale (UQOL)

| | | | Active | Inter |
|---|---|---|---|---|
| | | Placebo | aspurũs | Group |
| Parameter | Visit | (N = 33) | (N = 35) | p-Value |
| Occupational | Baseline Visit | 20.91 | 21.91 | 0.2063 |
| QoL | Visit 4 (Day 30 + 2) | 23.76 | 24.06 | 0.7249 |
| | Visit 5 (Day 60 + 2) | 22.73 | 29.20 | <0.0001 |
| Health QoL | Baseline Visit | 17.70 | 18.14 | 0.5678 |
| | Visit 4 (Day 30 + 2) | 19.30 | 25.14 | <0.0001 |
| | Visit 5 (Day 60 + 2) | 18.30 | 25.60 | <0.0001 |
| Emotional QoL | Baseline Visit | 21.33 | 20.63 | 0.2751 |
| | Visit 4 (Day 30 + 2) | 17.09 | 25.83 | <0.0001 |
| | Visit 5 (Day 60 + 2) | 17.97 | 27.31 | <0.0001 |
| Sexual QoL | Baseline Visit | 10.15 | 10.29 | 0.7993 |
| | Visit 4 (Day 30 + 2) | 9.67 | 12.40 | <0.0001 |
| | Visit 5 (Day 60 + 2) | 7.97 | 13.80 | <0.0001 |
| Total QoL | Baseline Visit | 70.09 | 70.97 | 0.5161 |
| | Visit 4 (Day 30 + 2) | 69.82 | 87.43 | <0.0001 |
| | Visit 5 (Day 60 + 2) | 66.97 | 95.91 | <0.0001 |

TABLE 7
Summary Statistics of Dass 21 Questionnaire

| | | | Active | Inter |
|---|---|---|---|---|
| | | Placebo | aspurũs | Group |
| Parameter | Visit | (N = 33) | (N = 35) | p-Value |
| Depression | Baseline Visit | 29.88 | 30.51 | 0.5708 |
| | Visit 4 (Day 30 + 2) | 31.52 | 20.57 | <0.0001 |
| | Visit 5 (Day 60 + 2) | 33.27 | 14.00 | <0.0001 |
| Anxiety | Baseline Visit | 31.70 | 30.34 | 0.0938 |
| | Visit 4 (Day 30 + 2) | 32.85 | 20.91 | <0.0001 |
| | Visit 5 (Day 60 + 2) | 36.06 | 13.60 | <0.0001 |
| Stress | Baseline Visit | 31.52 | 31.89 | 0.6868 |
| | Visit 4 (Day 30 + 2) | 33.27 | 21.89 | <0.0001 |
| | Visit 5 (Day 60 + 2) | 34.12 | 14.97 | <0.0001 |

### D. Hot Flashes and Night Sweats

A five-point Likert scale was used to measure these secondary outcomes of hot flashes and night sweats. Hot flashes are the most common menopausal symptom which is a sudden feeling of warmth in the body. In the placebo group, out of 33 participants, 19 (57.57%) experienced hot flashes by the final visit (visit 5), whereas, in the active group, out of 35 participants, only four (12.12%) experienced hot flashes by the final visit (visit 5).

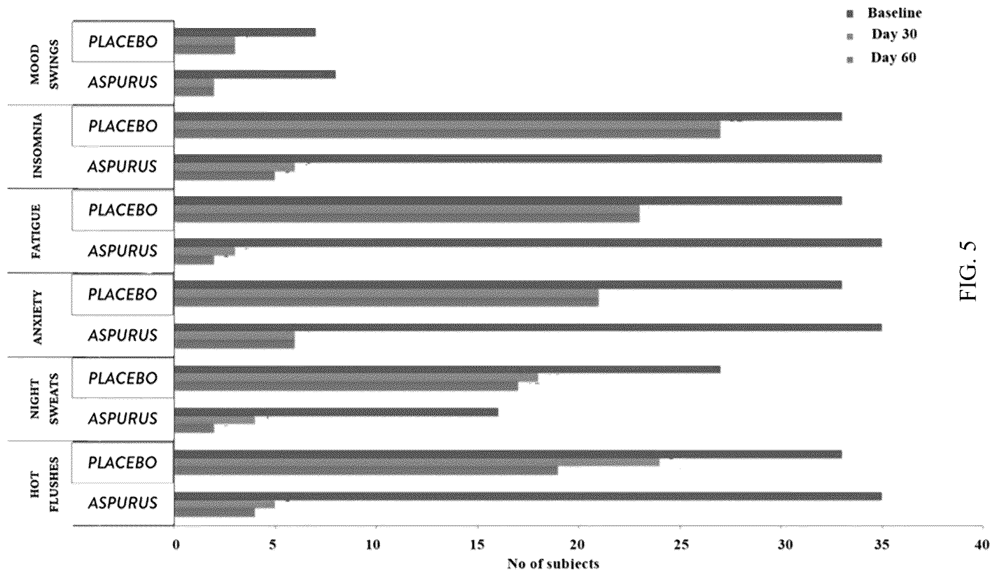

As shown in Table 8 and FIG. 5, the absolute mean number of hot flashes in the placebo group reduced from 2 per day at the baseline visit to 1.18 per day at the final visit (visit 5). On the other hand, in the active group, it reduced from 1.97 per day to 0.14 per day. This reduction of hot flashes from 1.83 per day in the active group compared to 0.82 per day in the placebo group clearly shows that the active group is in therapeutic response compared to the placebo group.

As shown in Table 8 and FIG. 5, there was a significant change (p<0.0001) in the hot flashes observed in the intergroup comparison at the baseline visit evaluation to the final visit (visit 5). This shows that most participants had a significant reduction in both the frequency and number of hot flashes per day.

Further, there was a significant positive change in the night flashes with a comparative intergroup p-value of 0.002 at the baseline visit to the final visit (visit 5) outcomes (p<0.0001). Hence, most participants had a significant reduction in both the frequency and number of night sweats per day.

The absolute mean number of anxiety/nervousness/feelings of worrying in the placebo group reduced from 2.36 per day at the baseline visit to 1.06 per day at the final visit (visit 5). On the other hand, in the active group, it reduced from 2.57 per day to 0.34 per day. This reduction of anxiety/nervousness/feeling of worrying from 2.23 per day in the active group compared to 1.3 per day in the placebo group suggests that the active group was a better therapeutic responder compared to the placebo group.

As shown in Table 8 and FIG. 5, there was a significant change in the anxiety/nervousness/feeling of worrying with a comparative intergroup p-value of 1 at the baseline visit to the outcome assessment at the last visit (visit 5) (p<0.0001). Therefore, the outcomes suggest that most participants had a significant reduction in both the frequency and number of anxiety/nervousness/feeling of worrying episodes per day. Moreover, the intergroup comparison of “how much did it affect your health status” was also statistically significant from the baseline visit results to the final visit (visit 5) outcomes.

TABLE 8
Summary Statistics of Likert Scale

| | | | Active | Inter |
|---|---|---|---|---|
| | | Placebo | aspurũs | Group |
| Parameter | Visit | (N = 33) | (N = 35) | p-Value |
| Hot Flushes | Baseline Visit | 33 | 35 | 1 |
| | Visit 4 (Day 30 + 2) | 24 | 5 | <0.0001 |
| | Visit 5 (Day 60 + 2) | 19 | 4 | <0.0001 |
| Night Sweats | Baseline Visit | 27 | 16 | 0.0026 |
| | Visit 4 (Day 30 + 2) | 18 | 4 | <0.001 |
| | Visit 5 (Day 60 + 2) | 17 | 2 | <0.0001 |
| Anxiety/ | Baseline Visit | 33 | 35 | 1 |
| Nervousness/ | Visit 4 (Day 30 + 2) | 21 | 6 | 0.0027 |
| Feeling of Worry | Visit 5 (Day 60 + 2) | 21 | 6 | <0.0001 |
| Fatigue/General | Baseline Visit | 33 | 35 | 1 |
| Weakness with | Visit 4 (Day 30 + 2) | 23 | 3 | <0.0001 |
| Tiredness | Visit 5 (Day 60 + 2) | 23 | 2 | <0.0001 |
| Insomnia/ | Baseline Visit | 33 | 35 | 1 |
| Sleeplessness/ | Visit 4 (Day 30 + 2) | 27 | 6 | <0.0001 |
| Disturbed Sleep | Visit 5 (Day 60 + 2) | 27 | 5 | <0.0001 |

As shown in Table 8, the absolute mean number of fatigues in the placebo group reduced from 2.64 per day at the baseline visit to 1.03 per day at the final visit (visit 5). On the other hand, in the active group, it reduced from 2.69 per day to 0.06 per day. This reduction of fatigue from 2.63 per day in the active group compared to 1.61 per day in the placebo group suggested that the active group had a better therapeutic response compared to the placebo group. Further, a significant change in fatigue with a comparative intergroup p-value of 1 at the baseline visit to the final evaluation outcomes (p<0.0001) was recorded at the final visit (visit 5). Hence, the outcomes indicate that most participants had a significant reduction in both the frequency and the number of fatigue episodes per day.

Similarly, insomnia/sleeplessness/disturbed sleep, mood swings/sudden changes of mood loss of libido, and urinary incontinence significantly reduced in the active group when compared to the placebo group. Therefore, the treatment group had a better outcome for the less affected sleep, improved interpersonal relationships, and improved sexual relationships with their partners. Vaginal dryness that causes the sensation of burning/itching of the vagina was experienced by fewer study participants in the active group than in the placebo group. The results were similar for bladder weakness and urinary incontinence where fewer participants experienced them in the active group.

As shown in FIG. 5, the treatment satisfaction was better in the active group than the placebo group which can be attributed to the positive effects of the test product evaluated in the present study for menopausal symptoms namely, mood swings, insomnia, fatigue, anxiety, hot flashes, and night sweats.

### E. Safety Results

As shown in Table 9, overall, nine participants reported adverse events, with five participants from the active group having experienced six adverse events (dizziness and bloating) and four from the placebo (MCC) group having experienced five adverse events (bloating and nausea). However, all reported adverse events were mild, and all vital parameters were within the physiological range at all visits. Moreover, it was observed that none of the adverse events were mild. Hence, no action was taken on the study treatment administration because of the adverse events. The vital signs and other observational data were measured as part of the clinical study. Systolic blood pressure, diastolic blood pressure, pulse rate, body temperature, and respiratory rate were measured. There were no significant changes in the above vitals in both groups.

TABLE 9
Summary of Adverse Events for all Subjects

Count (N = 68)

| | | |
|---|---|---|
| | PLACEBO | ACTIVE aspurũs |
| PARAMETER | (N = 33) | (N = 35) |

Have patients experienced Adverse Events?

| | | |
|---|---|---|
| Yes | 5 (15.15%) | 4 (11.43%) |
| No | 28 (84.84%) | 31 (88.57%) |

If yes, specify

| | | |
|---|---|---|
| Dizziness | 0 (0.00%) | 1 (2.86%) |
| Bloating | 5 (15.15%) | 4 (11.43%) |
| Nausea | 1 (3.03%) | 0 (0.00%) |

### F. Conclusion

The findings from this study support the positive effects of a novel Shatavari formulation taken for 60 days at a dose of 250 mg twice a day. This study also showed that the test product was safe and well-tolerated with no major adverse events in the participants with menopausal symptoms (hot flashes, anxiety, stress, sleep, etc.). Therefore, it could be a safe alternative to modern drugs. The efficacy of the test product was well demonstrated concerning a significant improvement in menopausal symptoms. It was found to be an effective therapy for psychological and somatic problems related to menopausal syndrome.

The findings of our study supported the traditional use of Shatavari with modern scientific evidence. Further, clinical and pharmacological studies with longer durations and larger sample sizes are required to understand the mechanistic actions of this formulation in menopausal women.

We claim:

1. A method to extract shatavarin I and shatavarin IV from roots of A. racemosus to increase the concentration of the total shatavarin I and shatavarin IV in the extract, wherein,
the method comprises the steps of
a) adding roots of A. racemosus in a solvent;
b) stirring the roots in the solvent at 90° C. for maceration;
c) filtering the macerated mixture to separate the residue from the filtrate;
d) repeating steps a)-c) for at least 3 more times to obtain the final residue;
e) evaporating the residue under vacuum at a boiling point of the solvent to obtain dried extract comprising of shatavarin I and shatavarin IV.

2. The method as claimed in claim 1, wherein, the total concentration of shatavarin I and shatavarin IV is in the range of 4-6.5% of the extract.

3. The method as claimed in claim 1, wherein, the solvent used for extraction of shatavarins from roots of A. racemosus is selected from the group consisting of mixture of methanol and water, and mixture of ethanol and water.

4. The method as claimed in claim 1, wherein, the solvent used for extraction of shatavarins from roots of A. racemosus is mixture of methanol and water at a ratio selected from the group consisting of 80:20, 70:30, 60:40, 50:50, 40:60.

5. The method as claimed in claim 1, wherein, the solvent used for extraction of shatavarins from roots of A. racemosus is mixture of methanol and water at a ratio of 70:30.

6. The method as claimed in claim 1, wherein, the solvent used for extraction of shatavarins from roots of A. racemosus is mixture of ethanol and water at a ratio selected from the group consisting of 80:20, 70:30, 60:40, 50:50, 40:60.

7. The method as claimed in claim 1, wherein, the solvent used for extraction of shatavarins from roots of A. racemosus is mixture of ethanol and water at a ratio of 70:30.

8. The method as claimed in claim 1, wherein, the shatavarin I and shatavarin IV extract has effect in managing menopausal symptoms in women including reduction in hot flashes, night sweats, insomnia, anxiety, nervousness, vaginal dryness, and loss of libido.
